(19) United States
(12) Patent Application Publication
(10) Pub. No.: US 2021/0154104 A1
(43) Pub. Date: May 27, 2021

(54) SUBSTANCES AND METHODS FOR BEDSIDE MANAGEMENT OF POST-OPERATIVE PAIN

(71) Applicant: Hila Yousefi, Stockholm (SE)

(72) Inventors: Hila Yousefi, Tehran (IR); Mahoud Torabinejad, Laguna Niguel, CA (US)

(73) Assignees: Hila Yousefi, Stockholm (SE); Mahmoud Torabinejad, Laguna Niguel, CA (US)

(21) Appl. No.: 16/693,232

(22) Filed: Nov. 23, 2019

(51) Int. Cl. A61K 6/69; A61K 31/416; A61K 47/36; A61K 31/65; A61K 47/12; A61K 47/22; A61K 6/52

(52) U.S. Cl. CPC A61K 6/69; A61K 31/416; A61K 47/36; A61K 6/52; A61K 47/12; A61K 47/22; A61K 31/65

(57) ABSTRACT

A solution for treating post-operative local pain, removing debris and bacteria from tooth surfaces for use in general dentistry and in endodontics. The solution compromising analgesic, disinfectant, surfactant, organic acid, and sustained release polymer. A method of making a solution for treating post-operative pain, removing debris and bacteria from tooth surfaces for use in general dentistry and in endodontics. A solution and method for use in general dentistry and in endodontics to treat post-operative local pain in oral mucosa.

### INCORPORATION BY REFERNCE TO ANY PRIORITY APPLICATIONS

This application is a continuation of U.S patent application Ser. No. 10/348,298, filed on Jan. 21, 2003, the entire contents of which are hereby incorporated by reference and made a part of this specification.

### BACKGROUND

Mixture of tetracycline, acid and detergent (MTAD) (sold under the trade name Biopure MTAD (Dentsply Tulsa Dental, Tulsa, Okla., USA) is a substance currently used in general dentistry and in endodontics to remove smear layer from prepared tooth surface. While mixture of tetracycline, acid and detergent has proven to be biocompatible and suitable for removing smear layer from prepared tooth surfaces, mixture of tetracycline, acid and detergent is not capable for management of post-operative pain. This post-operative pain requires a patient to take analgesic drugs for few days after treatment increasing the cost and inconvenience of treatment. Therefore, there is a need for a solution to both remove smear layer from prepared tooth surface and that is not subject to this disadvantage.

### SUMMARY

The present invention relates to methods for treating post-operative pain. The methods inhibit the activity of inflammatory mediators in procedures such as root canal treatment, dental reconstruction, periodontal procedures, and the like. A solution compromising mixture of tetracycline, acid, detergent, analgesic, and slow release polymer is used in the method. The surfactant is selected from a class of the non-ionic emulsifiers used in pharmaceuticals and food preparation. Preferably, the disinfectant is tetracycline or doxycycline, the acid is citric acid, the analgesic is benzydamine hydrochloride, and the sustained-release polymer is chitosan.

Such solutions are useful in a multitude of dental treatment applications, including, but not limited to, root canal therapy; reconstructive dentistry such as direct and indirect pulp caps, crowns, bridges, veneers, and the like; other endodontic procedures; and periodontic procedures. Such solutions also are useful for treating local post-operative pain in surgical procedures.

### DETAILED DESCRIPTION OF THE PREFERRED EMBODIMENTS

As used in this disclosure, “post-operative pain” refers to pain resulting from irritation of pulpal or periradicular tissues leading to inflammation. Pulp irritants can be broadly classified as nonliving or living. Nonliving causes of inflammation may be mechanical, thermal, or chemical. Viable irritants include microorganisms and viruses. This pain is of inflammatory origin. Microorganisms present in dental caries are the main source of irritation of the dental pulp and periradicular tissues. Mechanical irritants, such as deep cavity preparations, removal of tooth structure without proper cooling, deep periodontal curettage, may lead to alterations in the underlying pulp. Chemical irritants of the pulp include various dentin cleansing, sterilizing, and desensitizing substances, in addition to some of the substances present in temporary and permanent restorative materials and cavity liners.

As used in this disclosure, “disinfectant” refers collectively to compositions that are able to suppress or eliminate bacterial or other microorganisms found in endodontic or periodontic sites. The term “disinfectant” includes antibiotics and antimicrobials as these terms are understood in pharmaceutical science.

As used in this disclosure, “analgesic” refers to collectively to compositions that are able to achieve analgesia, and relief from pain as in endodontic or periodontic sites. The term “disinfectant” includes antibiotics and antimicrobials as these terms are understood in pharmaceutical science.

As used in this disclosure, “sustained-release polymer” refers to collectively to biodegradable controlled-release polymers and polymeric nanoparticles, The term “sustained-release polymer” includes polymers designed to release a drug at a predetermined rate in order to maintain a constant drug concentration for a specific period of time with minimum side effects as these terms are understood in pharmaceutical science.

The solution of this invention comprises disinfectant, analgesic, sustained release polymer, surfactant, and organic acid. In a preferred embodiment, the disinfectant is an antibiotic. It will be apparent to one skilled in the art that the antibiotic should be stable in the acidic solutions of which it forms a part, should be compatible with the other components of the solution, and should retain its effectiveness for at least the time of preparation of the solution and its application and residence time on or in the prepared tooth or bone surface. Examples of such antibiotics include, but are not limited to, ansamycins, including rifamycins; cephalosporin; macrollides Such as clarithromycin,josamycin, and oleandomycin; most polypeptides, such as bacitracin, capreomycin, enduracidin, enviomycin, gramicidin, mikamycin, ristocetin, thiostrepton, tyrocidine, viomycin, and Virginiamycin; all tetracycline compounds. such as apicycline, chlortetracycline, clomocycline, demeclocycline, doxycycline, guamecycline, lymecycline, mecleocycline, methacycline, minocycline, oxytetracycline, penimepicycline, pipacycline, rollitetracycline, sancycline, mupirocin, and tetracycline-HCl; and tuberin. Most quinolones such as ciprofloxacin, gatifloxacin, and moxifloxacin are not preferred, as they are weak bases and have decreased effect in acidic solutions. Additionally, most B-lactam antibiotics, particularly penicillins, are also not preferred, as they are generally unstable in acidic solutions. Exceptions, however, are amoxycillin, an acid-stable member of the penicillin family, and similar compounds.

Tetracyclines are broad-spectrum antibiotics that are effective against a wide range of microorganisms. They include tetracycline-HCl, minocycline, and doxycycline. Tetracyclines are bacteriostatic in nature and are generally more effective against gram-positive bacteria compared to gram-negative bacteria. A reference to tetracycline shall be taken to include all members of the tetracycline family. A number of Studies have shown that tetracyclines significantly enhance healing after Surgical periodontic therapy. Members of the family of tetracyclines are preferred for use herein. Tetracyclines are preferred for a number of reasons. One reason they are preferred is because they have many unique properties along with their antimicrobial effect. For example, tetracycline-HCl has a low pH in concentrated solution and thus can act as a calcium chelator, and cause enamel and root Surface demineralization. Tetracycline HCl's surface demineralization of dentin is comparable to that seen using citric acid. In addition, it has been shown that tetracycline-HCl is a Sustentative medication and becomes absorbed and released from tooth structures such as dentin and cementum. The use of tetracycline is also preferred because a very low portion of the general population exhibits allergies or other sensitivities to tetracycline. Doxycycline, a broad-spectrum antibiotic synthetically derived from oxytetracycline, is particularly preferred. Doxycycline is available as doxycycline monohydrate, doxycycline hyclate; doxycycline hydrochloride hemiethanolate hemihydrate; and doxycycline calcium for oral administration.

In another preferred embodiment, the disinfectant is an antimicrobial compound. It will be apparent to one skilled in the art that the antimicrobial compound should be stable in the acidic solutions of which it forms a part, should be compatible with the other components of the solution, and should retain its effectiveness for at least the time of preparation of the solution and its application and residence time on or in the prepared tooth or bone surface. Examples of such antimicrobial compounds include, but are not limited to, chlorhexidine compounds. Chlorhexidine gluconate is preferred. One example of a suitable chlorhexidine gluconate solution is a commercially available 0.12% solution known as “PeridexTM”. Additionally, the use of chlorhexidine gluconate has been found to be especially desirable in patients who exhibit sensitivity or allergy to tetracycline compounds.

It will also be recognized by one skilled in the art that the analgesic used should be stable in acidic solution with an antibiotic compound. Preferable is an analgesic that provides analgesic and anti-inflammatory effects. Additionally preferable is an analgesic that acts locally, thus providing an increased analgesic effect and permitting enhanced pain relief and anti-inflammatory treatment into dentinal tubules, periradicular tissue and irregular spaces that are difficult to reach by analgesics that cannot act locally.

In a preferred embodiment, the analgesic is a locally-acting nonsteroidal anti-inflammatory drug with local anaesthetic and analgesic properties for pain relief and anti-inflammatory treatment of inflammatory conditions, preferably one commonly used in for treating inflammatory pain in the mouth and throat.

In the improved solution of this invention, the analgesic used is selected from the group of nonsteroidal anti-inflammatory drugs. One exemplary member of the preferred class used in for treating inflammatory pain in the mouth and throat is benzydamine (available as the hydrochloride salt).

It will also be recognized by one skilled in the art that the detergent used should be stable in acidic Solution with an antibiotic compound. Additionally preferable is a detergent that reduces Surface tension of the Solution, thus providing an increased wetting effect and permitting enhanced penetration of the irrigation Solution into dentinal tubules and irregular spaces that are otherwise difficult to reach. Furthermore, the detergent should be one suitable for use in situ in dental applications without deleterious effect to the human or animal subject.

In a preferred embodiment, the detergent is a non-ionic Surfactant or Similar compound, preferably one commonly used in the food and drug industry or approved for use by the Food and Drug Administration. Examples of such compounds include, but are not limited to, mono- and di-glycerides; sucrose esters, sorbitan esters (also known as SPANs), particularly sorbitan monostearate; sorbitols; polysorbates (polyoxyethylene Sorbitan esters, also known in industry as TWEENs), particularly polysorbate 20, polysorbate 60, polysorbate 65, and polysorbate 80; Stearoly lactylates, lecithin and derivatives, polyglycol fatty acid esters, p-Cymene; quaternary ammonium compounds, Sodium alkyl Sulfonates, triethanolamine; and alkyl polysaccharides.

In another preferred embodiment, the detergent used is selected from the group of sorbitan esters or polysorbates. One exemplary member of the preferred class is polysorbate 80 (polyoxyethylene Sorbitan monooleate).

It will also be apparent to one of skill in the art of dentistry that the acid used should be suitable for dental application. Thus, the acid should be nontoxic in the applicable concentration and amount used in the irrigation process and should also be compatible with the detergent and disinfectant selected as the other components of the solution. Preferred acids must also be capable of dissolving the organic and inorganic components of the Smear layer within the chosen exposure time, but without inducing unwanted erosion of the tooth and Surrounding Surfaces.

In another preferred embodiment, the acid is an organic acid, preferably having pKa values between 1.5 and 5. Further preferred are carboxylic acids or other acids with a polar nature and pKa values between 2 and 5. In a further preferred mode of the present invention, an acid with a pKa value between about 2.75 and 3.75 is used. One exemplary member of the preferred class is citric acid. Citric acid is particularly suitable when tetracycline is chosen as the disinfectant, because citric acid does not diminish or otherwise alter the antibacterial effect of tetracycline.

It will be apparent to one skilled in the art, however, that stronger acids may also be preferred for use in the present invention provided that the time of application of the solution is shortened accordingly. As such, stronger acids including, but not limited to, chloracetic, maleic, saccharic, tartaric, and polyacrylic may be used, having pKa values ranging from about 0.5 to about 3.0. Mixtures may also be used. In some embodiments inorganic acid, specifically phosphoric acid may find utility so long as the essential properties of the solution are maintained.

It will also be apparent to one of skill in the art of dentistry that the sustained release polymer used should be suitable for dental application. Thus, the sustained release polymer should be nontoxic in the applicable concentration and amount used in the irrigation process and should also be compatible with the acid, analgesic, detergent and disinfectant selected as the other components of the solution. Preferred sustained release polymer must also be capable of releasing the analgesic for prolonged period of time to maintain a constant analgesic concentration.

In a preferred embodiment, the sustained release polymer is an organic polymer preferably one commonly used in the food and drug industry or approved for use by the Food and Drug Administration. Examples of such compounds include, but are not limited to poly lactic-co-glycolic acid, poly-L-lactic acid, chitosan.

The disinfectants are present in the solutions of the present invention in weight percentages of from about 1 to about 5 percent of the Solution and preferably in amounts of from about 2 to about 4 weight percent, with amounts of about 3 percent being even more preferred, especially when the disinfectant is a tetracycline.

The analgesic is preferably present in the solutions of the invention in weight percentages of from about 0.5 to about 1.0 percent of the solution, with amounts of from about 0.75 to about 1.0 percent being more preferred. Amounts by weight of about 0.75 percent are generally most preferred, especially when the detergent is benzydamine hydrochloride.

The detergent is preferably present in the solutions of the invention in weight percentages of from about 0.1 to about 1.5 percent of the solution, with amounts of from about 0.25 to about 1.0 percent being more preferred. Amounts by weight of about 0.5 percent are generally most preferred depending upon the detergent, especially when the detergent is a polysorbate.

The acids of the invention are present in the solutions in amounts of from about 0.5 to about 10 percent by weight of the solution, preferably from about 3 to about 6 percent. More preferred are solutions having weight percentages of acid, especially organic acid, of from about 4 to about 5 percent.

The sustained release polymer is preferably present in the solutions of the invention in weight percentages of from 0.1 to about 1.5 percent of the solution, with amounts of from about 0.25 to about 0.5 percent, especially when the polymer is chitosan with medium molecular weight.

In general, the solutions of the invention are acqueous and water comprises the bulk of the balance of the composition. Solutions of the invention may also include other compounds, however, so long as they do not interfere with the essential functions of the principal components, do not cause them to degrade and do not interfere with the convenience and utility thereof. Such additional additives may include colorants, flavorants, stabilizers, and other materials conventionally added to dental solutions. One particularly useful adjuvant may be chelating agents capable of rendering chelatable materials, especially metals, soluble. Indeed, use of a polyfunctional acid may achieve this goal. It will be recognized by one of skill in the art that regardless of the components or additives in the solution, the resulting Solution should be sterile so that the objectives of the invention are achieved. In all cases, such materials are present in effective amounts to accomplish their objectives.

In a preferred embodiment of the current invention, the solution comprises an aqueous solution of 3% doxycycline, 0.75% benzydamine Hcl, 0.5% polysorbate 80, 4.25% citric acid, and 0.25% chitosan by weight. While these components have previously been used separately and in high concentrations in efforts to remove the smear layer and relief inflammatory pain, the five components as described above have not been combined as in the present invention. Additionally, studies performed in conjunction with the present invention using a solution of 3% doxycycline, 0.75% benzydamine Hcl, 0.5% polysorbate 80, 4.25% citric acid, and 0.25% chitosan have shown low levels of cytotoxicity and no mutagenicity when compared to all-purpose bleach, which had previously been used to disinfect tooth preparations.

The present invention is directed to methods for achieving analgesia and relieving from post-operative pain in a design to release the drug at a predetermined rate in order to maintain a constant drug concentration for a specific period of time, sterilizing and removing the smear layer on a prepared tooth or canal surface comprising irrigating the surface with a solution comprising disinfectant, analgesic, surfactant, acid, and sustained release polymer. In preferred modes of the invention, the analgesic is a nonsteroidal anti-inflammatory drug that is sufficiently stable in an acidic environment. The substance releases the drug at a slow rate in order to maintain a constant drug concentration for a specific period of time. A study was performed to determine the analgesic effect of adding benzydamine Hcl to mixture of tetracycline, acid and detergent (MTAD).

Experiments were performed on male Sprague-Dawley rats (n=15, 200-220 g) housed at 23±1° C. and 12-h light/dark cycles, acclimatized to the laboratory conditions for at least 72 h before use, with free access to food and water. Tests were carried out during the light phase (between 10:00 am and 5:00 pm) in a silent room. All solutions were administered by Subcutaneous (SC) injection (40 μL). The solution prepared by mixing doxycycline, citric acid, Tween 80, Benzydamine Hcl, chitosan and compared with the mixture of 3% doxycycline, 5% citric acid, 0.5% Tween 80 as control. Animal assigned into three groups (5 animals in each group):
1.3% doxycycline, 5% citric acid, 0.5% Tween 80 (MTAD)
2.3% doxycycline, 5% citric acid, 0.5% Tween 80, 0.5% Benzydamine Hcl, 0.25% Chitosan,
3.3% doxycycline, 5% citric acid, 0.5% Tween 80, 0.75% Benzydamine Hcl, 0.25% Chitosan

SC injection of dilute formalin has been shown to produce inflammatory pain. The orofacial formalin test was performed following standard protocol. That is, before the injection, each rat was placed in a transparent plexiglass observation chamber (30×30×30 cm3 with a mirror placed at an angle of 45°) for 30 min, in order to minimize stress-related behaviors. SC injection of 40 μL 2.5% formaline was performed followed by injection of 40 μL test materials into the upper lip, just lateral to the nose, using a 30-gauge sterile needle. The rats were immediately returned to the transparent box for a 30-minute observation. Rubbing of the injected area was regarded as the parameter of nociceptive response. Duration of nociceptive response was cumulatively recorded using a stopwatch, in consecutive 1-min intervals over a 30-minute period, and was considered as an index of nociception. Response scoring was performed according to accepted protocol. This score was based on four scales including 0 for normal behavior e.g. grooming; 1.abnormal head movements; 2.abnormal continuous shaking of the lower jaw; 3.excessive rubbing of the mouth. Referring now to Table 1, there are shown the average scores of five rats every 1 minute over 30 minutes observation.

TABLE 1

| | Test Group 1 | Test Group 2 | Test Group 3 |
|---|---|---|---|
| Time | (Average scores | (Average scores | (Average scores |
| (Minute) | of 5 rats) | of 5 rats) | of 5 rats) |
| 1 | 0 | 0 | 0 |
| 2 | 0.25 | 1.125 | 0.40 |
| 3 | 0.50 | 0.75 | 0.65 |
| 4 | 0.75 | 0.90 | 0.65 |
| 5 | 0.50 | 0.55 | 0.45 |
| 6 | 1 | 0.60 | 0.60 |
| 7 | 1.25 | 0.85 | 0.90 |
| 8 | 1.50 | 1.05 | 0.70 |
| 9 | 1.50 | 0.85 | 0.75 |
| 10 | 1.50 | 1 | 0.50 |
| 11 | 1.25 | 0.50 | 0.65 |
| 12 | 1.25 | 0.75 | 0.60 |
| 13 | 2 | 0.85 | 0.80 |
| 14 | 2 | 0.45 | 0.30 |
| 15 | 2 | 0.40 | 0.50 |
| 16 | 1.25 | 0.55 | 0.60 |
| 17 | 2.50 | 0.30 | 0.35 |
| 18 | 2.5 | 1.15 | 0.65 |
| 19 | 2.75 | 0.90 | 0.90 |
| 20 | 2.25 | 0.40 | 0.20 |
| 21 | 2.25 | 0.70 | 0.50 |
| 22 | 2.25 | 0.60 | 0.55 |
| 23 | 1.75 | 0.70 | 0.50 |
| 24 | 1.75 | 0.90 | 0.70 |
| 25 | 2 | 0.45 | 0.30 |
| 26 | 1.50 | 0.30 | 0.25 |
| 27 | 2 | 0.45 | 0.20 |
| 28 | 2.25 | 0.20 | 0.20 |
| 29 | 1.75 | 0.50 | 0.45 |
| 30 | 2.5 | 0.45 | 0.30 |

As can be seen, mixture of 3% doxycycline, 5% citric acid, 0.5% Tween 80, 0.75% Benzydamine Hcl, 0.25% chitosan induces pain reduction in comparison with MTAD.

In one embodiment of the present invention, the disinfectant is an antibiotic that is sufficiently stable in an acidic environment. It is further preferred that the antibiotic be a tetracycline compound. More preferably, the tetracycline compound is doxycycline, particularly in the form of doxycycline hyclate.

In another preferred aspect of the present invention, the acid is an organic acid, preferably having a pKa between 1.5 and 5. In a further preferred embodiment, the organic acid has a pKa between 2 and 4; preferably between 2.75 and 3.75, such as that of citric acid. In a further embodiment, the acid is phosphoric acid.

According to another embodiment of the present invention, there is provided a method of making a substance for use in general dentistry and in endodontics to manage post-operative pain with simultaneous antibacterial effect and remove smear layer from tooth surface , where the substance release in a period of time. The method comprises benzydamine Hcl as an analgesic and chitosan as a sustained release polymer.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference in their entirety.

1. A method of bedside management of post-operative pain in general dentistry and in endodontics:
a)Mixing an analgesic with any compositions and methods for irrigating a prepared dental root canal.
b)Making use of slow-sustained release polymer for longer analgesic and antibacterial effects of any compositions and methods for irrigating a prepared dental root canal.

2. A substance for bedside management of post-operative pain in general dentistry and in endodontics:
a)Adding benzydamine Hcl as an analgesic and chitosan as a sustained release polymer to mixture of tetracycline, acid and detergent.

1. The method of claim 1, wherein the method does not involve using slow-sustained release polymer.

2. The method of claim 1, wherein the method relieving pain longer period of time.

3. The method of claim 1, wherein the method relieving more severe pain.

4. The method of claim 1, wherein the analgesic is not benzydamine Hcl
The method of claim 1, wherein the slow-sustained release polymer is not chitosan.
